(19) United States
(12) Patent Application Publication
(10) Pub. No.: US 2010/0256559 A1
(43) Pub. Date: Oct. 7, 2010

(54) Device for the intermittent occlusion of the coronary sinus

(75) Inventors: Werner Mohl, Altenmarkt-Thennenberg (AT); Jon H. Hoem, Oberageri (CH)

Correspondence Address: FISH & RICHARDSON P.C., PO BOX 1022, MINNEAPOLIS, MN, 55440-1022, US

(73) Assignee: MIRACOR Medical Systems GmbH, Vienna (AT)

(21) Appl. No.: 12/454,258

(22) Filed: May 14, 2009

(30) Foreign Application Priority Data
Apr. 3, 2009 (AT) A 536/2009

(51) Int. Cl. A61M 25/092

(52) U.S. Cl. 604/95.04; 604/95.01

(57) ABSTRACT

In a device for the intermittent occlusion of the coronary sinus, including a catheter with several lumens and an occlusion device fixed to the catheter and operable through at least one lumen of the catheter, the operating elements of the catheter cooperate with a first axial portion of the catheter for bending the same in said first portion, and with at least one further axial portion of the catheter for bending the same in said at least one further axial portion, and/or for stiffening said axial portions.

Priority is claimed to Austria Pat. App. A536/2009, filed Apr. 3, 2009, hereby incorporated by reference.

The invention relates to a device for the intermittent occlusion of the coronary sinus, including a catheter with several lumens and an occlusion device fixed to the catheter and operable through at least one lumen of the catheter.

Arterial blood, which supplies the heart muscle, is able to pass through healthy heart tissue while nourishing the same, yet has difficulty reaching ischemic tissue. As a result, the supply of ischemic tissue with nutrients and the discharge of metabolic catabolites from such ischemic tissue will be impaired.

In this context, it has already been proposed to supply the ischemic tissue with blood through retrograde perfusion. To this end, it was proposed to use an inflatable balloon fixed to the end of a catheter to intermittently occlude the coronary sinus. The blood pressure in the coronary sinus rises during the occlusion at every heart beat so as to cause blood reaching the coronary sinus through the healthy tissue of the heart muscle to be flushed back into the ischemic tissue. For such an intermittent coronary sinus occlusion, the balloon end of the catheter is inserted either percutaneously or surgically. The other end of the catheter is supplied with a gas or fluid by a pump, which causes the cyclic inflation and deflation of the balloon. A device for the retroinfusion of coronary veins is, for instance, known from WO 2005/120602, by which a pressure-controlled, intermittent coronary sinus occlusion can be performed. In that device and the associated method for determining the optimum times for triggering and releasing the occlusion, pressure parameters like the speeds of the pressure increase and pressure drop were determined and subjected to relatively complex processing.

For the percutaneous insertion of the catheter, it is proceeded in a manner that the catheter is guided via the inferior caval vein or the superior caval vein into the right atrium of the heart, into which the coronary sinus runs. Due to the position of the mouth of the superior caval vein or inferior caval vein, respectively, relative to the mouth of the coronary sinus, the introduction of the catheter into the coronary sinus requires considerable skill from the surgeon in order to direct the tip of the catheter into the coronary sinus in such a manner as to enable the subsequent introduction of the catheter along with the occlusion device. It frequently happened that several attempts of introduction into the coronary sinus were required, thus considerably extending the duration of treatment and, hence, the strain on the patient.

It is, therefore, the object of the present invention to further develop a device of the initially specific kind to the effect that the introduction of the catheter into the coronary sinus is facilitated, while additionally enabling enhanced anchoring of the catheter against relative displacement by the dynamic pressure of the blood in the coronary sinus during the occlusion.

To solve this object, a device of the initially defined kind is further developed to the effect that operating elements of the catheter cooperate with a first axial portion of the catheter for bending the same in said first portion, and with at least one further axial portion of the catheter for bending the same in said at least one further axial portion, and/or for stiffening said axial portions. The fact that the catheter can be bent by operating elements in specific axial portions enables the surgeon, after having reached the right atrium, to deflect the catheter from the direction of the mouth of the caval vein used to access and orient the same to the mouth of the coronary sinus, whereby a precise controllability will be obtained on account of the catheter being bendable by the operating elements in at least one further axial portion as in correspondence with the invention. The planes in which the catheter can be bent in the various axial portions may be identical or different and are adapted to the anatomical features of the respectively chosen method for introducing the catheter. Once the catheter with the occlusion device is located in the desired position within the coronary sinus, stiffening of the axial portions by the operating elements can prevent the catheter from slipping out on account of the pressure building up during occlusion, since the stiffened catheter, which follows the vessels, will come into abutment on the vessel walls in the curves and will thereby be secured against slipping.

When introducing the catheter into the coronary sinus, it is above all important to direct the tip of the catheter, i.e. the most distal portion of the catheter, into the desired vessels, since, once the tip has reached the correct vessel and, in particular, the coronary sinus, the remaining catheter will relatively easily follow the insertion. The invention is, therefore, advantageously further developed to the effect that the first axial portion and the at least one further axial portion are arranged distally to the occlusion device in the region of the distal end of the catheter. After the distal end or tip of the catheter has been pushed into the right atrium via the superior or inferior caval vein, the surgeon is able to effect, by the aid of the operating elements, a first deflection of the catheter tip from the direction of the mouth of the respective caval vein such that the tip will be directed to the mouth of the coronary sinus in the right atrium, wherein further suitable bending in a further distally located axial portion of the catheter tip will cause the shape of the catheter to adapt to the extension course of the coronary sinus.

In order to be able to reliably orient the catheter from the mouth direction into the atrium to the mouth and the direction of the coronary sinus, precise controlling is necessary. The invention is, therefore, advantageously further developed to the effect that the operating elements cooperate with the deformable axial portions mechanically, pneumatically and/or electrically.

The anchorage of the catheter in the desired position within the coronary sinus against the dynamic pressure occurring during occlusion will be the more reliable, the longer the stiffened portion following the vessels. Besides, said anchorage will be the gentler the longer the stiffened portion, since the anchoring force results from the sum of the abutment forces in the curves of the vessels, which is why the individual abutment points will be relieved in the event of a large number of abutment points. The invention is advantageously further developed to the effect that the operating elements of the catheter, for stiffening the catheter, cooperate with axial portions substantially over the entire axial length of the catheter.

According to a preferred embodiment of the present invention, the catheter according to the invention is further developed to the effect that Bowden pulls are provided for bending and/or stiffening the catheter, which constitutes a particularly intuitive and precisely controllable option of bending the catheter in specific axial portions. In such an embodiment, Bowden pulls can be arranged in lateral lumens of the catheter and fixed to a counter support in a desired axial portion.

After the catheter has been bent, with the bending strength of the catheter material opposing the tensile force of the catheter by a certain counter force, it is feasible to secure the induced bend by fixing the Bowden pull in the respective position and thereby stiffen the catheter.

According to a further preferred embodiment, the invention is further developed to the effect that pneumatic means are provided for bending and/or stiffening the catheter. In such an embodiment, it is conceivable to feed specific axial catheter portions, which are made of a material that is more elastic than the rest of the catheter, with pressure medium via separate lumens, thus causing a relative elongation of the flexible catheter portion to which pressure is fed and, hence, bending. Due to the bending strength of the catheter material, stiffening will again be achieved, if the pressure is maintained over an extended period of time.

According to a further preferred embodiment of the present invention, the configuration is devised such that memory-shape materials are provided for bending and/or stiffening the catheter. Memory-shape materials can, for instance, be inserted as rods in the outer wall of the catheter and traversed by direct current via electrical contacts. These materials are characterized by changing their dimensions and, in particular, their lengths in the event of rod-shaped parts, which will again cause folding of the catheter. In this case too, stiffening of the catheter is realized in that the memory-shape-material components changed in terms of length exert a pressure or tension force against the restoring force of the elastic catheter, thus stiffening the catheter during the maintenance of direct current.

According to a particularly preferred embodiment of the present invention, stiffening of the catheter is effected independently of the bending mechanism and is accomplished in that an electrorheological fluid is provided for stiffening the catheter, which electrorheological fluid may be provided in a separate lumen of the catheter. As the catheter is being brought into the desired configuration and position by the aid of the operating elements, the viscosity of the fluid can be increased by the application of direct current through appropriately arranged contacts on the ends of the fluid column in the lumen, which will altogether lead to the stiffening of the catheter over its entire length.

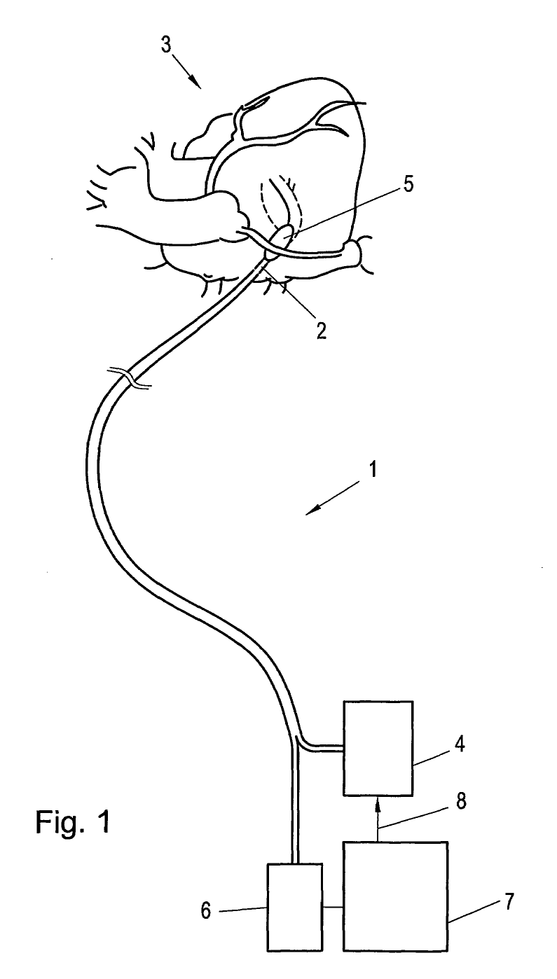

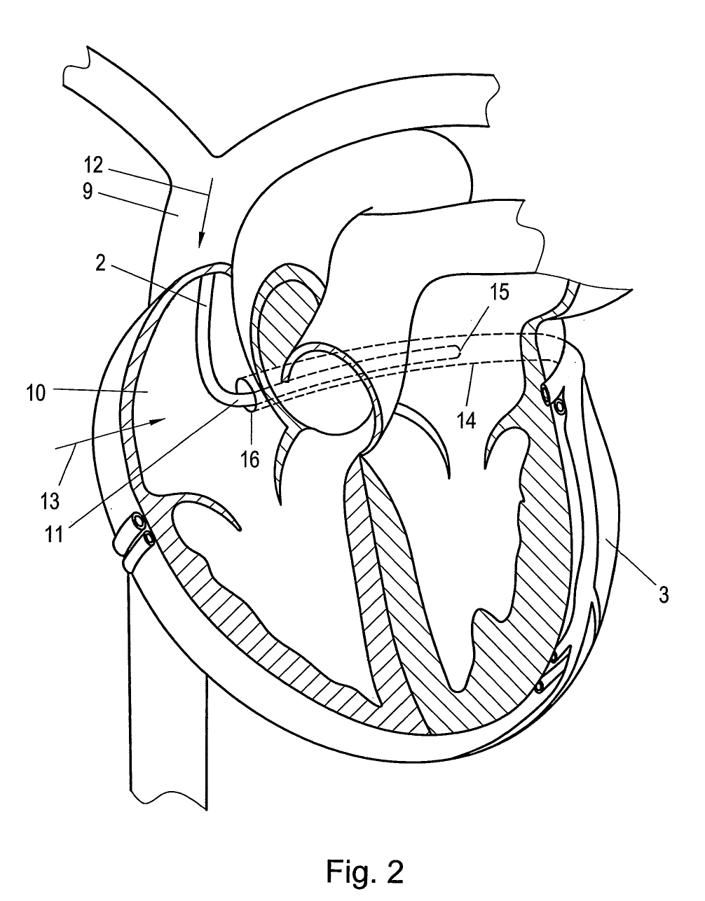

In the following, the invention will be explained in more detail by way of an exemplary embodiment schematically illustrated in the drawing. Therein, FIG. 1 is a schematic view of a heart comprising a device for the intermittent occlusion of the coronary sinus according to the present invention; and FIG. 2 is a partial, sectional view of a heart, with a catheter entering from the superior caval vein into the right atrium and being introduced into the coronary sinus under bending.

In FIG. 1, the device for the intermittent occlusion of the coronary sinus is schematically denoted by 1, wherein a multi-lumen catheter 2 is apparent, whose distal end is inserted in the coronary sinus of the heart 3 via the right atrium. One lumen of the catheter 2 is connected with a pump 4, which provides pressure medium for inflating the occlusion device 5. The pressure in the coronary sinus is acquired by a pressure measuring device 6, said pressure measuring device 6 also including a memory for the detected measurements. The respectively measured pressure values are fed to a control device comprising an evaluation circuit 7 to deliver control signals via line 8 for starting and stopping the pump 4.

In FIG. 2, a heart is again denoted by 3, with the catheter 2 running into the atrium 10 via the superior caval vein 9. In the axial portion 11, the catheter 2 can be bent by operating elements of the catheter 2 and thus directed from the mouth direction indicated by arrow 12, of the superior caval vein into the mouth direction indicated by arrow 13, of the coronary sinus 14. When the tip 15 of the catheter 2 has reached the mouth 16 of the coronary sinus 14, the surgeon may reduce the adjustment force by the aid of the operating elements so as to enable the catheter 2 to slide into the coronary sinus 14. Once the catheter 2 is located in the desired position within the coronary sinus 14, and the occlusion device 5 is, in particular, completely inserted in the coronary sinus, the surgeon may again apply the respective adjustment forces for anchoring the catheter 2 in the coronary sinus 14 by the aid of the operating elements so as to prevent the catheter 2 from being pushed out by the dynamic pressure in the coronary sinus 14.

1. A device for intermittent occlusion of a coronary sinus, comprising:
a catheter comprising a plurality of lumens, and
an occlusion device fixed to the catheter and operable through at least one of the lumens of the catheter,
wherein operating elements of the catheter cooperate with a first axial portion of the catheter for bending the catheter in said first axial portion, and with at least one second axial portion of the catheter for bending the catheter in said at least one second axial portion, and/or for stiffening one or more of said first axial portion and said at least one second axial portion.

2. A device according to claim 1, wherein the first axial portion and said at least one second axial portion are arranged distally to the occlusion device in a region of a distal end of the catheter.

3. A device according to claim 1, wherein the operating elements cooperate with the first axial portion and said at least one second axial portion one or more of mechanically, pneumatically, and electrically.

4. A device according to claim 1, wherein the operating elements of the catheter cooperate with said first and at least one second axial portions substantially over an entire axial length of the catheter, to stiffen the catheter.

5. A device according to claim 1, further comprising Bowden pulls positioned for one or more of bending and stiffening the catheter.

6. A device according to claim 1, further comprising pneumatic means positioned for one or more of bending and stiffening the catheter.

7. A device according to claim 1, further comprising memory-shape materials positioned for one or more of bending and stiffening the catheter.

8. A device according to claim 1, further comprising an electrorheological fluid provided in one of said lumens to effect stiffening of the catheter.

9. A device according to claim 2, wherein the operating elements cooperate with the first axial portion and said at least one second axial portion one or more of mechanically, pneumatically, and electrically.

10. A device according to claim 2, wherein the operating elements of the catheter cooperate with said first and at least one second axial portions substantially over an entire axial length of the catheter, to stiffen the catheter.

11. A device according to claim 3, wherein the operating elements of the catheter cooperate with said first and at least one second axial portions substantially over an entire axial length of the catheter, to stiffen the catheter.

12. A device according to claim 2, further comprising Bowden pulls positioned for one or more of bending and stiffening the catheter.

13. A device according to claim 3, further comprising Bowden pulls positioned for one or more of bending and stiffening the catheter.

14. A device according to claim 4, further comprising Bowden pulls positioned for one or more of bending and stiffening the catheter.

15. A device according to claim 2, further comprising pneumatic means positioned for one or more of bending and stiffening the catheter.

16. A device according to claim 3, further comprising pneumatic means positioned for one or more of bending and stiffening the catheter.

17. A device according to claim 4, further comprising pneumatic means positioned for one or more of bending and stiffening the catheter.

18. A device according to claim 5, further comprising pneumatic means positioned for one or more of bending and stiffening the catheter.

19. A device according to claim 2, further comprising memory-shape materials positioned for one or more of bending and stiffening the catheter.

20. A device according to claim 3, further comprising memory-shape materials positioned for one or more of bending and stiffening the catheter.
